(19) United States
(12) Patent Application Publication
(10) Pub. No.: US 2020/0155848 A1
(43) Pub. Date: May 21, 2020

(54) LIGHT THERAPY DEVICE AND METHOD

(71) Applicant: Lightening Health LLC, Rochester Hills, MI (US)

(72) Inventor: Rhonda Sousley, Rochester Hills, MI (US)

(73) Assignee: Lightening Health LLC, Rochester Hills, MI (US)

(21) Appl. No.: 16/689,770

(22) Filed: Nov. 20, 2019

Related U.S. Application Data
(60) Provisional application No. 62/917,130, filed on Nov. 21, 2018.

(51) Int. Cl. A61N 1/36; A61N 5/06

(52) U.S. Cl. CPC A61N 1/36036; A61N 5/0613; A61N 2005/0647; A61N 2005/0651; A61N 2005/0626; A61N 1/36031

(57) ABSTRACT

The portable light therapy device comprises a housing, at least one energy generations source, for generating energy such as light and electricity, the device further comprising at least one probe for contacting a user and providing stimulation to a selected location on the user. The device may be wearable, portable, and may further have wired and unwired communication capability. The device may be used on or around the ear, and is further customizable. The method steps of use of the device contemplate either user operation or operation by another to locate stimuli locations on a user, position the light therapy device and further operate the device to provide desired stimulation and adapt to feedback from the device.

### CROSS-REFERENCE TO RELATED APPLICATION

This application claims priority to U.S. Provisional Application No. 62/917,130, filed Nov. 21, 2018, and which is hereby incorporated by reference in its entirety.

### FIELD OF THE DISCLOSURE

The present invention relates generally to a light therapy device and method to treat stimulus points in the ear. More particularly, the invention consists of a device with flexible aspects, and a method for, positioning light and electrical sources in exact stimulus points accounting for differing ear shapes and sizes.

### BACKGROUND OF THE DISCLOSURE

Currently there are a number of solutions for stimulation of auricular points. Some of these solutions attempt to utilize needles, magnets, or electricity, but these solutions fail to meet the needs of the industry because they are not safe, precise wearable devices that can be used by a nonprofessional or inexperienced user. These devices mainly use various points on the body rather than limit their treatment to the ear. Other solutions seek to utilize hand held light probe devices, but these solutions also fail to meet the industry needs because they require a practitioner to hold the light probe device in one stationary, non-changing point for a predetermined time duration and then relocate the device to another location, and repeat the process for the duration of the treatment. Still other solutions attempt to utilize light to treat specific ear stimulus points but these solutions are similarly unable to meet the needs of the industry because they do not provide for the flexibility of precise feedback to the user that the device is accurately placed and operating effectively. The disclosed invention solves the problems discussed above.

### SUMMARY OF THE DISCLOSURE

The present invention relates to a device and method associated with the device. With respect to the device, it is a light therapy device which may be worn, and has a power source, for example it may be battery operated, and provides multiple or fixed wavelengths of energy, over multiple or fixed time cycles, and contacts a user and stimulates various points on the body which include the ear, with light, electricity, or in combination stimulation thereof. The device is adjustable and can be used to position light and electrical sources in predetermined stimulus point locations determined by the user accounting for differing sized and shaped ears.

The core components of one embodiment of the invention are a wearable housing with a power source which may be rechargeable, including a battery-operated controller and adjustable positioning light probe arms which have the ability to be positioned as follows: a programmable controller contained in a C-shaped hook to secure the device over and behind the ear, or in a hand-held control device, and adjustable wire light probe arms to transmit light, electrical, and sensory stimulation to predetermined points located on the ear.

In general the device contemplated by the invention includes: a housing; a power source; an energy-generation source; an electronic processor; an electronic controller; and an energy-emitting probe having a length, wherein said probe is movable as connected to the device, is connected on a first end to at least one other element of the light therapy device, and is configured to emit energy through a second end, wherein said second end is configured to contact at least one stimulus point of a user that is external to said user's body.

In this application, having a length may also include no length between a probe and the device.

As contemplated by the invention, when an embodiment of the invention includes more than one probe, said probes may be independently movable.

The invention contemplates use of movable arms which may span the distance between the first end and second end. Further, the invention contemplates multiple probes, which may have moveable arms which are configured to span the distance between the first end and second end of the probe.

Said movable arm may be substantially self-sturdy, such that when positioned by a user said arm remains substantially in place.

For this application, light emitting and transmitting sources and probes include, but are not limited to light emitting diodes (LEDs), laser diodes, intense pulse lights (IPLs), and other light sources. Light generating, emitting, and transmitting sources and may be adapted to pulse according to a proprietary algorithm that is programed into the memory of a control device.

For this application, electrical emitting or transmitting sources include, but are not limited to probes, wires, and other current carrying elements. The electricity generating, emitting, and transmitting sources may be adapted to pulse according to a proprietary algorithm that is programed into the memory of a control device.

For this application, sensory stimulation emitting or transmitting sources include, but are not limited to probes, wires, and other current carrying elements. The sensory stimulation generating, emitting, and transmitting sources may be adapted to pulse according to a proprietary algorithm that is programed into the memory of a control device.

Some of the surfaces of the external components of the device may be constructed of and encompass an elastic type material, examples of which include elastomeric-based material, silicone-based material, among others. This elastic type material may provide comfort while wearing the device as well as overall protection of the device. The light and electrical emitting source of the device is controlled by at least one electronic processor which has the ability or possesses the characteristics to generate electrical stimulation, and emit light or electrical energy in various wavelengths, including visible light, infrared light and ultraviolet light, as well as control the time cycle and duration of the therapy session or both or all of these characteristics.

With respect to the associated method of use, the following steps are included: for each specific condition at least one stimulus point is determined including at least one wavelength, frequency, and time cycle. The device is positioned over the ear and the light and electricity emitting adjustable arms are positioned to a location on the predetermined points. The programmable settings of at least one electronic processor are adjusted accordingly for predetermined stimulation of the ear points. At the conclusion of these steps the user experiences a mild electrical feedback to confirm that the device is properly positioned and is operational and therefore receives safe an effective stimulation of the ear points for the treatment of a condition.

It is desirable to have a comfortable and safe wearable device that can be utilized by an inexperienced or non-professional user to treat various conditions through the precise placement of light and electricity on user selected auricular points. Further, it would also be desirable to have a device that can accommodate precise placement on points of differing ear shapes and sizes. Still further it would be desirable to have a device that can be programmed for varying frequencies, wavelengths, and time cycles. The disclosed device and associated method advantageously fill these needs and addresses the deficiencies by providing a safe and effective ear acupuncture treatment device.

The light energy source or sources are capable or emitting the light energy at a power sufficient to achieve the predetermined subsurface power density based on outputs of a programmable controller, based on setting selected by a user. Depending on the condition to be treated, the corresponding wave lengths, intensity levels, pulse rate, and time interval, among other parameters for the treatment are received by a control system and emitted by the device's light source.

In another embodiment, a customizable ear insert for insertion into the outer ear or ear canal or both of a user, with one or more LED/laser lights, where the lights can be specifically placed in strategic places for the needs of the individual user. The lights will directly and comfortably contact the body. Impressions can be taken of the user's ear to produce customized ear molds which can then be utilized to manufacture perfectly fitting individualized inserts.

In another embodiment, a user's ear can be scanned and a digital representation formed in a suitable computer application program which can be used to produce a customized ear insert.

The device may also have one or more of the following: a battery that is disposable or rechargeable, USB port for charging, additional light probe arms, wifi wireless controls, corded hand held controls, a power supply that can plug into a standard wall outlet (ie, a 110 volt outlet) electrodes for electrical stimulation, electrical sensors for user feedback through an app, and sensory stimulation and electrical sensors to provide feedback of proper placement of stimulation.

Communication with the device from the controller may be via wifi, Bluetooth, or other wireless controls. Similarly, communication with the device may be via a wired connections such as those known in the art of wired communications.

Power sources connected to the device may be likewise through a wired or wireless connection. When wired connections are employed, the power source may be a battery that is worn behind the ear or attached to a cord for comfortable placement at a distance, remote from the ear.

As used in the patent, a processor may also process an algorithm, interface with a controller of the invention and provide necessary outputs to one or more energy emitting probes, where the energy omitted is in one or more forms, such as electrical, audio, mechanical or other forms of energy. Said processor may be configured to communicate with at least one of a controller or energy emitting probe.

As used in the patent, a controller may also communicate settings after being interfaced with by a user or outside control device, such as through wireless communication by wifi, Bluetooth, or otherwise. Said controller may be configured to communicate with at least one of a processor or energy emitting probe.

When equipped with more than one type of energy emitting probe, such as electrical, mechanical, audio or otherwise, one or more of the energy emitting probes may be configured not to contact a user and instead be configured to emit feedback to the user to confirm proper placements and operation of the device. As addressed in the method of the invention, said user may further modify the positioning of at least one energy emitting probe in contact with the user to achieve feedback of proper function of the device. When equipped with an energy emitting probe for emitting electrical energy, said probe may be referred to as an electrode.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The light therapy device and method may, however, be embodied in many different forms and should not be construed as being limited to the embodiments set forth herein; rather, these embodiments are provided by way of illustration only and so that this disclosure will be thorough, complete, and fully convey understanding to those skilled in the art.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a perspective right-side view of a light device constructed in accordance with the invention, showing the device on a human ear.

FIG. 2 is a cross sectional view of light emitting adjustable arm. A portion has been cut away to facilitate illustration of the internal components.

FIG. 3 is a perspective back view of the light device of FIG. 1.

FIG. 4 is a partially broken away perspective view of an aspect of the light device housing.

FIG. 5 is a block diagram of a control circuit for the light device.

FIG. 6 is a block diagram of a control circuit for an alternate embodiment of the light device.

FIG. 7 is a perspective right-side view of an alternative embodiment wherein the insert is customized and manufactured to fit an individual ear. This embodiment shows a cord to an alternate battery source that is hand held.

FIG. 8 is a view of the customized ear insert with lights.

FIG. 9 is an exploded view of the customized embodiment

FIG. 10 is a view of a wearable device comprised of a plastic housing, two flexible probe arms, a behind-the-ear battery and battery housing, five probes in the form of LEDs, a USB port, a power button, audible feedback features and audio volume control.

FIG. 11 is an exploded view of a wearable device comprised of a plastic housing, two flexible probe arms, a behind-the-ear battery and battery housing, five probes in the form of LEDs, two adjustable members, a USB port, an audio source, a power button, audible feedback features and audio volume control.

### DETAILED DESCRIPTION OF THE PREFERRED EMBODIMENT

Referring to the Figures, wherein like numerals indicate like or corresponding parts throughout the several views, a device for light therapy is illustrated.

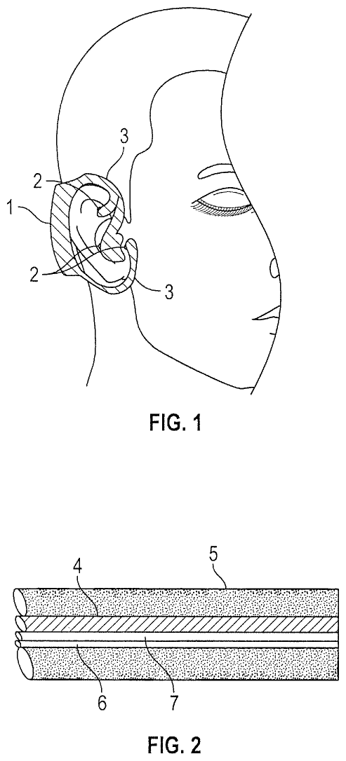

Now referring to FIG. 1, the core components of the invention are a wearable housing with a rechargeable power source and adjustable positioning light probe arms which have the ability to be positioned as follows: a programmable and rechargeable controller contained in a C-shaped hook housing (1), to secure the device over and behind the ear with adjustable wire light probe arms (3), to transmit light and electrical stimulation to predetermined ear points located on the ear through select points in the light probe (2).

Referring to FIG. 2, some of the surfaces of the adjustable arms can be constructed and encompass an elastic type material (5), examples include elastomeric based material, silicone-based material, among other elastic materials, to provide comfort while wearing the device as well as overall protection of the device. Contained inside the adjustable arms is an adjustable member (4), which can be configured to adjust the position of the arm, examples include a bendable material such as metal or similar. Associated with the adjustable member is a light emitting wire (6), common examples include a fiber optic cable. The adjustable member also has associated with it an electrical wire (7), to transfer electrical current throughout the adjustable arm.

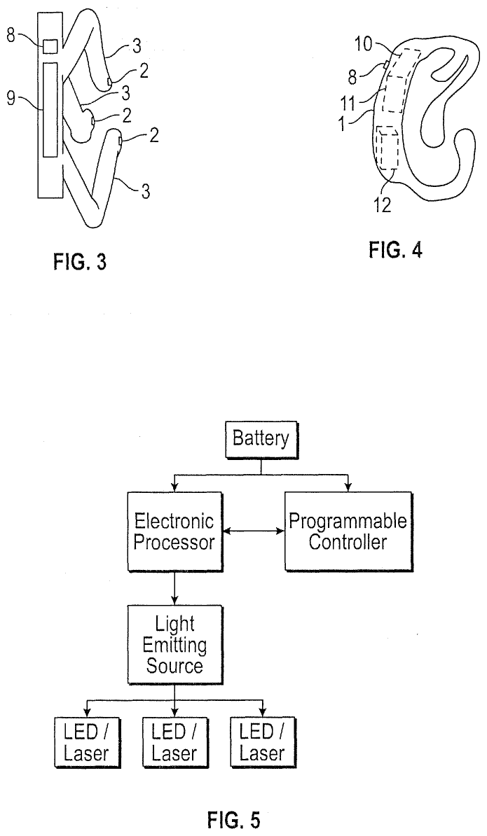

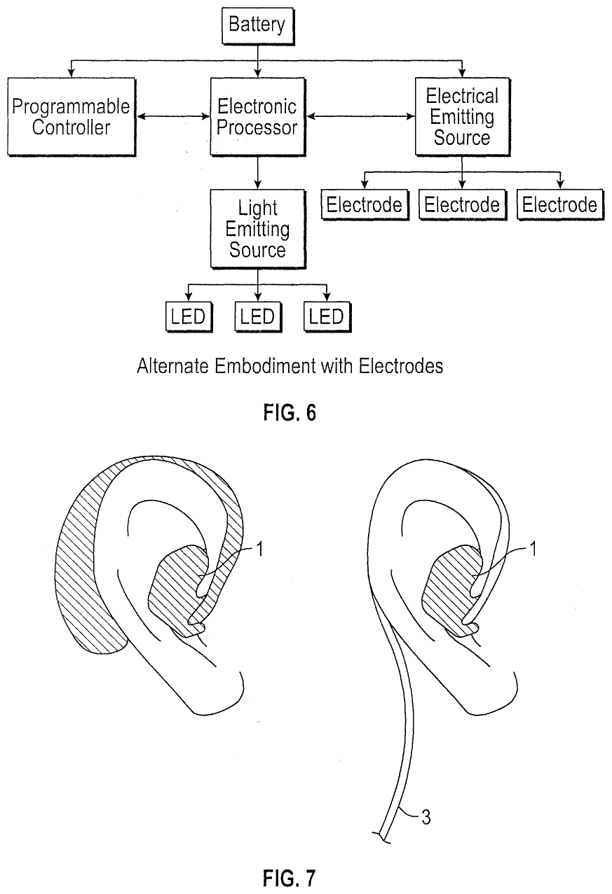

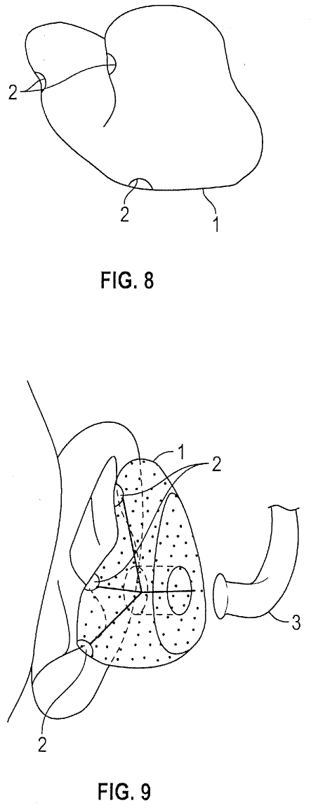

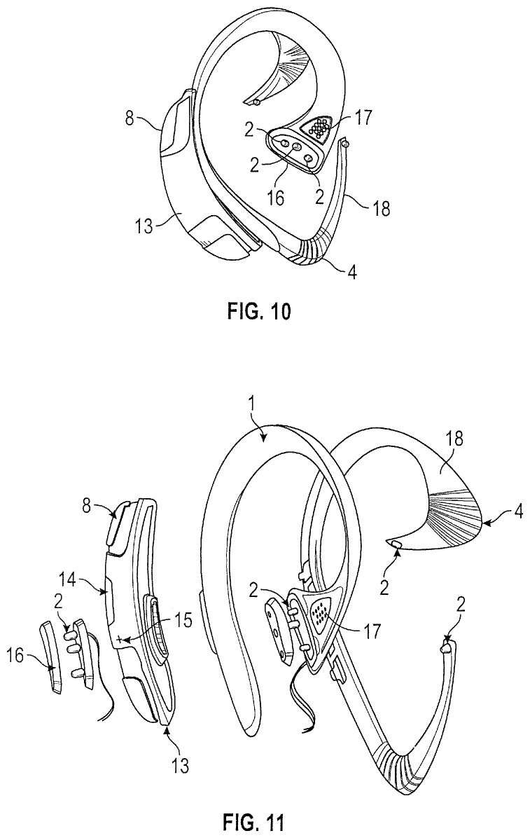

Referring to FIG. 3, the light therapy device houses a way for the user to turn the device on and off most commonly with an on/off switch (8). Also illustrated is a possible location for the programmable controller (9), for manual program operation.

Referring now to FIG. 4, the light therapy device (1), contains a light emitting source (10), which generates the light to be transferred to the light probe arms and in this embodiment, receives its power from the power source which contains a battery (12). It is well known in the art that a power source does not have to be a battery, but for illustration purposes in this embodiment, a battery was chosen. The power source is charged via standard charging methods which can include a USB port or other suitable means. The light and electrical emitting source of the device is controlled by at least one electronic processor (11), which has the ability or possesses the characteristics to generate electrical stimulation , light in various wavelengths, including visible, infrared, and ultraviolet, as well as the time cycle duration of the therapy session or both or all of these characteristics.

FIG. 11 shows an exploded view of a wearable device comprised of a C-shaped hook housing (1), two probe arms (18), a behind-the-ear battery and battery housing (13), five probes in the form of LEDs (2), a USB port (14), a power button (8), audible feedback features (17), a probe spacer (16), and audio volume control (15).

One embodiment of the invention may further include a transmitter which is configured to interface with a remote application on a device such as a cell phone or a computer. The application and transmitter may interact over wireless communication or wired communication. Wireless communication may occur via Bluetooth, WiFi, or some other wireless communication mechanism or system known in the art of wireless communication. Wire communication may occur via USB, micro USB, or other wired communication mechanism or system known in the art of wired communication.

The invention has been described in an illustrative manner and it is to be understood that the terminology which has been used is intended to be in the nature of words of description rather that of limitation. Obviously, many modification and variations of the present invention are possible in light of the above teachings. It is therefore, to be understood that within the scope of the appended class, wherein reference numerals are merely for convenience and are not to be in any way limiting, the invention maybe practice otherwise than as specifically described.

Different features, variations and multiple different embodiments have been shown and described with various details. What has been described in this application at times in terms of specific embodiments is done for illustrative purposes only and without the intent to limit or suggest what has been conceived is only one particular embodiment or specific embodiments. It is to be understood that this disclosure is not limited to any single specific embodiments or enumerated variations. Many modifications, variations, and other embodiments will come to mind of those skilled in the art, and which are intended to be and are in fact covered by both this disclosure. It is intended that the scope of this disclosure should be determined by a proper legal interpretation and construction of the disclosure, including equivalents, as understood by those of skill in the art relying upon the complete disclosure present at the time of filing.

What is claimed is:

1. A light therapy device comprising:
a) a housing;
b) a power source;
c) an energy-generation source;
d) an electronic processor;
e) an electronic controller; and
f) an energy-emitting probe having a length, wherein said probe is movable, is connected on a first end to at least one other element of the light therapy device, and is configured to emit energy through a second end, wherein said second end is configured to contact at least on stimulus point of a user that is external to said user's body.

2. A device as in claim 1, wherein said device further comprises at least two energy-emitting probes.

3. A device as in claim 1, further comprising lighting means at said second end.

4. A device as in claim 3, wherein said light means at said second end is further characterized as an LED.

5. A device as in claim 3, wherein said second end further comprises a sound node for transmitting air pressure differences.

6. A device as in claim 3, wherein said second end further comprises a temperature node for transmitting temperature differences.

7. A device as in claim 1, wherein said device comprises a second energy-omitting device which is configured to provide sensory feedback to the user.

8. A device as in claim 1, further comprising conductive means at said second end.

9. A device as in claim 8, wherein said conductive means is further characterized as means for conducting electrical energy.

10. A device as in claim 1, wherein said processor is configured to generate at least one wavelength and at least one cycle time.

11. A device as in claim 1, wherein said housing is wearable and is configured to be worn as an earpiece outside of the outer ear.

12. A device as in claim 1, wherein said housing is wearable and is configured to be worn as an earpiece inside of the outer ear and substantially outside of the ear canal.

13. A device as in claim 1, wherein said wearable housing is comprised of an elastomeric-based material.

14. A device as in claim 1, wherein said wearable housing is comprised of a silicone-based material.

15. A device as in claim 1, wherein said power source is a battery.

16. A device as in claim 1, wherein said power source is an alternating current power source.

17. A device as in claim 1, wherein said device further comprises a transmitter configured to interface with an application, said application being usable on a remote device.

18. A device as in claim 1, wherein said device further comprises a power control feature, an audio source, an audio control volume control, and a USB port.

19. A method of using a light therapy device comprising:
a) A light therapy device is positioned over an ear of a user;
b) The user selects a stimulus point outside of the ear;
c) A probe of the light therapy device is moved and positioned substantially in contact with the stimulus point;
d) Using a controller of the light therapy device, an electronic processor of the light therapy device is adjusted to a set point corresponding to the position of the probes and the selected stimulus point;
e) The light therapy device is activated by the user;
f) The user receives feedback from the light therapy device probes and adjusts the probes as needed to substantially contact the stimulus point.

20. A method as in claim 19 wherein said step for positioning a light therapy device is further comprised of positioning the light therapy device over an ear such that the ear substantially holds the light therapy device in position in proximity to the ear.

21. A method as in claim 19 wherein said step for using a controller of the light therapy device to adjust the electronic processor of the light therapy device further comprises adjusting the set-point of a variable from the group consisting of: light therapy device wavelength, light therapy device frequency, and light therapy device cycle time, and further adjusting such the set-point of said variable until the feedback is achieved from the device by the user.
